(19) United States
(12) Patent Application Publication
(10) Pub. No.: US 2019/0022303 A1
(43) Pub. Date: Jan. 24, 2019

(54) INTRAVENOUS DELIVERY LINE ORGANIZER

(71) Applicants: William Douglas Headlee, Sandy, UT (US); David K. Sharette, Sandy, UT (US); Elizabeth Steichen, Spanish Fork, UT (US)

(72) Inventors: William Douglas Headlee, Sandy, UT (US); David K. Sharette, Sandy, UT (US); Elizabeth Steichen, Spanish Fork, UT (US)

(21) Appl. No.: 16/040,038

(22) Filed: Jul. 19, 2018

Related U.S. Application Data
(60) Provisional application No. 62/534,334, filed on Jul. 19, 2017.
(60) Provisional application No. 62/625,430, filed on Feb. 2, 2018.

(51) Int. Cl. A61M 5/14; F16L 3/22

(52) U.S. Cl. CPC A61M 5/1418; A61M 2209/08; F16L 3/22

(57) ABSTRACT

An apparatus, system, and method are disclosed for intravenous delivery line organization. The apparatus includes a body and a retention channel that includes an open portion and at least one retention tab. The system includes a plurality of apparatuses, each apparatus including a body and a retention channel that includes an open portion and at least one retention tab. The method includes providing an apparatus that includes a body and a retention channel that includes an open portion and at least one retention tab; and placing an intravenous delivery line into the retention channel of the apparatus.

### CROSS-REFERENCES TO RELATED APPLICATIONS

This application claims the benefit of U.S. Provisional Patent Application No. 62/534,334 entitled “INTRAVENOUS DELIVERY LINE ORGANIZER” and filed on 19 Jul. 2017, for William Douglas Headlee, et al., which is incorporated herein by reference. This application further claims the benefit of U.S. Provisional Patent Application No. 62/625,430 entitled “INTRAVENOUS DELIVERY LINE ORGANIZER” and filed on 2 Feb. 2018, for William Douglas Headlee, et al., which is incorporated herein by reference.

### FIELD

This invention relates to intravenous delivery lines and more particularly relates to organization of intravenous delivery lines.

### BACKGROUND

In medical settings, intravenous delivery lines are often tangled or difficult to identify, which can cause problems for quick identification, adjustment, removal and addition of new intravenous delivery lines. Such problems can even create risks to patient treatment and safety. Thus, a solution is needed.

### SUMMARY

An apparatus, system and method for organization of intravenous delivery lines is disclosed.

The apparatus comprises a body; and at least one retention channel located in the body that retains an intravenous delivery line; wherein each retention channel comprises an open portion that allows placement of the intravenous delivery line into the retention channel, and at least one retention tab that extends partially across the open portion of that retention channel.

The system comprises a plurality of apparatuses, each apparatus comprising a body, and at least one retention channel located in the body that retains an intravenous delivery line, wherein each retention channel of each apparatus comprises an open portion that allows placement of the intravenous delivery line into the retention channel, and at least one retention tab that extends partially across the open portion of the retention channel.

The method comprises providing a first apparatus comprising a body, and a retention channel located in the body, wherein each retention channel of the first apparatus comprises an open portion that allows placement of a first intravenous delivery line into the retention channel, and at least one retention tab that extends partially across the open portion of the retention channel; and placing the first intravenous delivery line into the retention channel of the first apparatus.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a perspective view illustrating one embodiment of an intravenous delivery line organizer;

FIG. 2 is a perspective view illustrating one embodiment of an intravenous delivery line organizer;

FIG. 3 is another perspective view illustrating one embodiment of an intravenous delivery line organizer;

FIG. 4 is a detail view of a portion of a retention channel of one embodiment of an intravenous delivery line organizer;

FIG. 5 is a detail view of an end of a retention channel of one embodiment of an intravenous delivery line organizer.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, structures, or characteristics of the invention may be combined in any suitable manner in one or more embodiments. In the following description, numerous specific details are provided. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Any schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

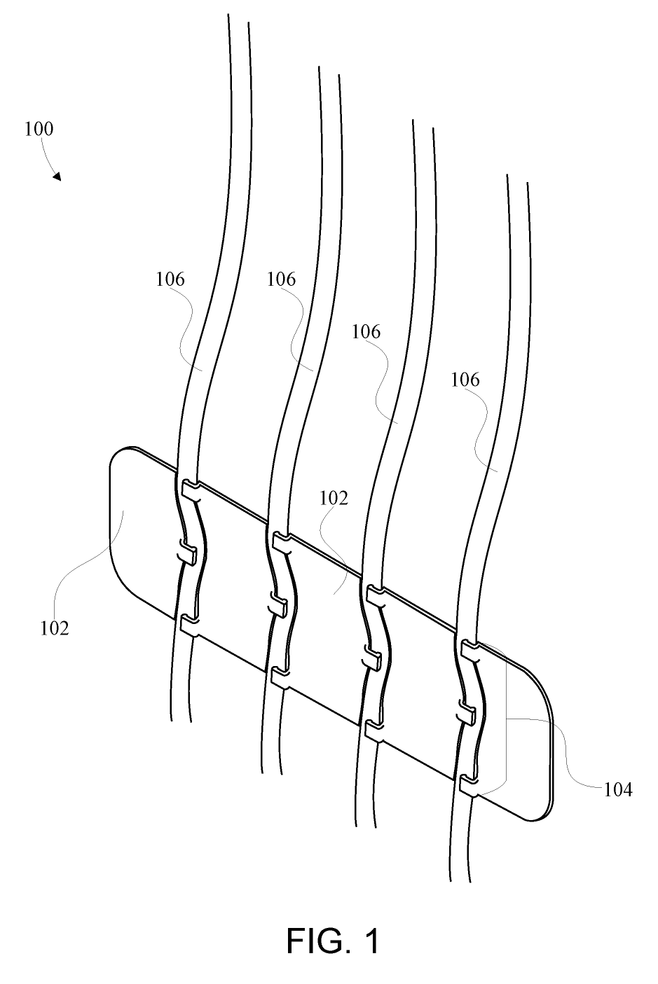

FIG. 1 is a perspective view illustrating one embodiment of an intravenous delivery line organizer.

In some embodiments, the apparatus 100 comprises a body 102 and a plurality of retention channels 104. A plurality of intravenous (IV) lines 106 run through the retention channels 104. The retention channels 104 retain and organize the IV lines 106 to prevent tangling, misidentification and other common problems in situations where multiple IV lines 106 are used.

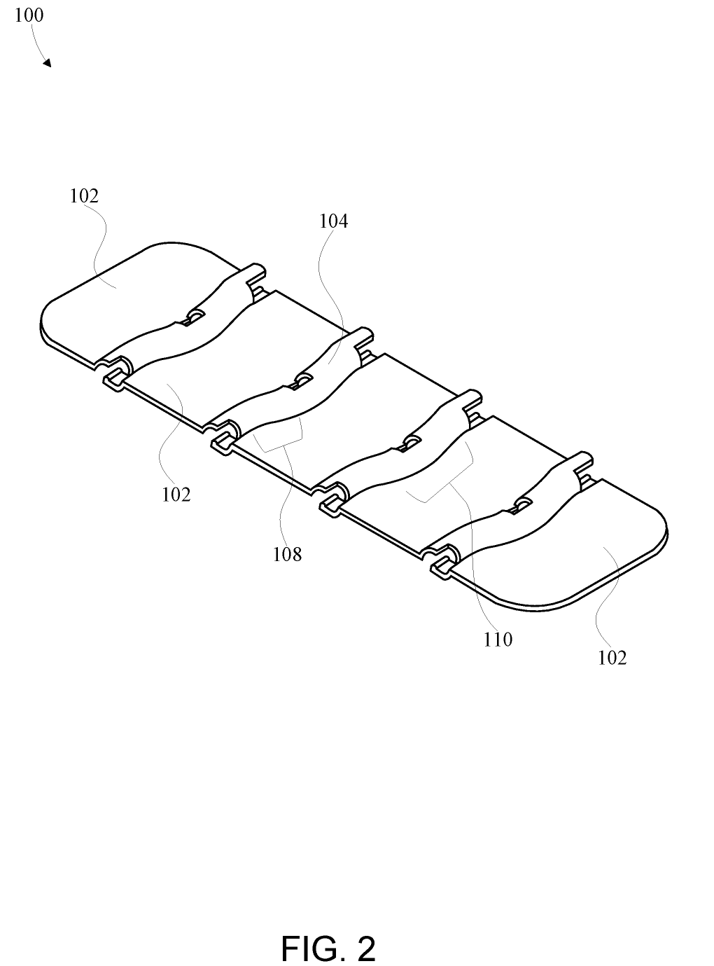

FIG. 2 is a perspective view illustrating one embodiment of an intravenous delivery line organizer.

In some embodiments, the body 102 is a flat, thin structure in one plane into which the retention channels 104 are placed. The body 102 comprises the portions of the apparatus 100 in between the retention channels 104, as well as the portions at either end of the apparatus 100. The body 102 couples and supports the retention channels 104 and provides surfaces for grasping the apparatus 100.

The apparatus 100 may be made of a variety of materials or combinations of materials, such as plastics, resins or metals. In some embodiments, the apparatus 100 may be made of a polypropylene plastic, which provides sufficient rigidity to support the IV lines 106 while providing a degree of flexibility to avoid breakage. In other embodiments, the apparatus 100 may be made of another material, such as ABS plastic, PVC plastic or other suitable material. The embodiments are not limited to specific configurations or examples provided herein.

In some embodiments, the entire apparatus 100 may be made of one type of material. In other embodiments, the apparatus 100 may be made of multiple materials. For example, the retention channels 104 may be made of one material, such as polypropylene plastic, and the body 102 may be made of another material, such as ABS plastic. Different materials will provide different properties that may be more desirable in certain situations. For example, if more flexibility is desired, a material that is more flexible than polypropylene may be used. The embodiments are not limited to the specific configurations or examples provided herein.

In some embodiments, the retention channels 104 have an inside diameter of four millimeters (4 mm), the same as the outside diameter of a standard IV line 106. In other embodiments, the retention channels 104 may have an inside diameter designed to fit outer diameters of other IV lines or other delivery lines, as necessary. The embodiments are not limited to the specific examples and configurations provided herein.

In some embodiments, the retention channels 104 comprise at least one bend 108 that provides additional friction on the IV lines 106 that helps retain the IV lines 106 without restricting the flow of fluids. In some embodiments, and as shown in FIG. 2, the retention channels 104 comprise a curved portion 110 that provides additional friction on the IV lines 106 and retains the IV line 106 that corresponds to each retention channel 104.

In some embodiments, such as the embodiment shown in FIG. 2, the apparatus 100 includes four retention channels 104. This number corresponds to the number of IV lines that are driven by a standard IV pump. However, other embodiments of the apparatus 100 include other numbers of retention channels 104. For example, some applications may only require two retention channels 104, whereas other applications may require ten or more. The embodiments are not limited to specific configurations or examples provided herein.

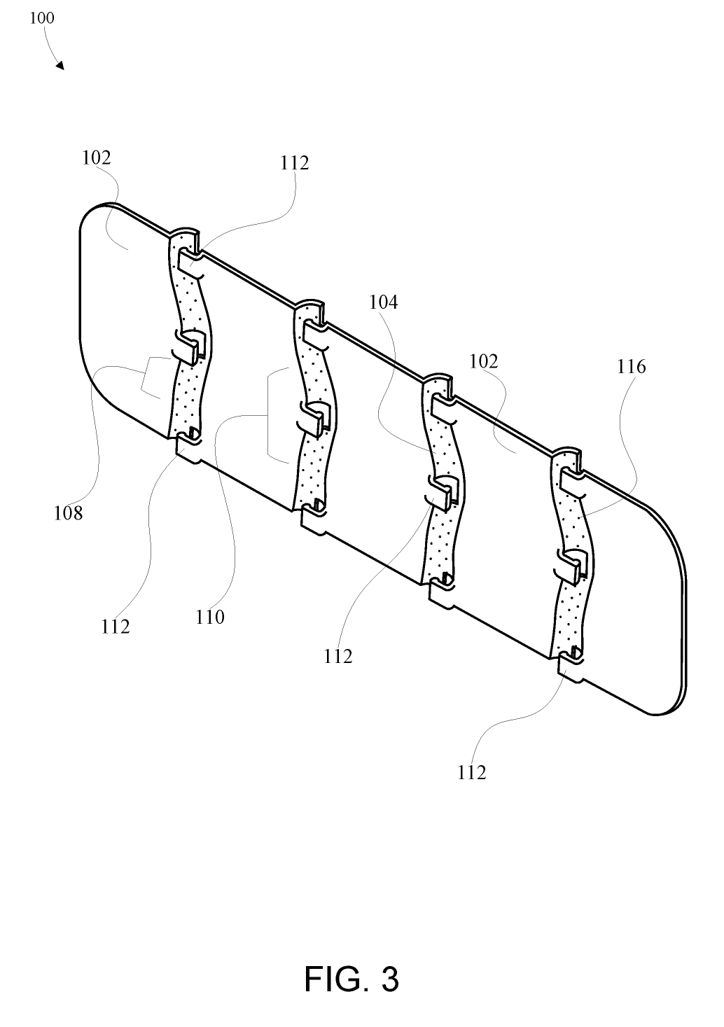

FIG. 3 is another perspective view illustrating one embodiment of an intravenous delivery line organizer.

Different embodiments of the apparatus 100 may comprise additional means of retention apart from the retention channels 104. Various methods of retention apart from the retention channels 104 are described herein; however, the embodiments are not limited to those specific configurations or examples provided. Additional methods of retention that do not depart from the spirit or essential characteristics of the embodiments fall within the scope of the present disclosure.

In some embodiments, the apparatus 100 additionally comprises retention tabs 112. The retention tabs 112 provide additional security to the IV lines, helping to retain the IV lines in the retention channel 104.

In some embodiments, the retention tabs 112 are positioned along the length of each retention channel 104 at regular intervals. In some embodiments, one retention tab 112 is placed approximately at the midpoint of the retention channel 104, and two more retention tabs are placed along the retention channel 104, one each towards each end of the retention channel 104. One example of such an arrangement is shown in FIG. 3.

In other embodiments, however, the apparatus 100 may comprise additional retention tabs 112 placed along the retention channel 104. Yet other embodiments may comprise fewer retention tabs 112. The embodiments are not limited to specific configurations or examples provided herein.

In some embodiments, each of the retention tabs 112 extends from the body to approximately one-half of the diameter of the retention channel 104. In other words, in such embodiments, each retention tab 112 extends halfway across the corresponding retention channel 104. Such an arrangement effectively retains the IV line 106 in the retention channel 104, while still allowing for easy placement and removal of the IV line 106. Other embodiments may include retention tabs 112 that extend further across the retention channels 104, and further embodiments may include retention tabs 112 that extend less than halfway across the retention channels 104. The embodiments are not limited to specific configurations or examples provided herein.

In some embodiments, the retention tabs 112 comprise rounded-off corners to avoid puncturing or damaging the IV lines 106, as well as to reduce the risk of user injury. In some embodiments, the retention channels 104 and the body 102 also comprise rounded-off corners and edges to avoid puncturing or damaging the IV lines 106 and reduce user injury.

In some embodiments, the retention channel 104 also includes retention nubs 116 that assist in retaining the IV line at least partially in the retention channel 104. Each retention nub 116 that contacts the IV line 106 applies an amount of pressure against the IV line, thus increasing the force that holds the IV line 106 in place. In some embodiments where a greater amount of retention is required, a greater number of retention nubs 116 may be included, thereby increasing the provided retention force. In embodiments requiring a lesser amount of retention, a fewer number of retention nubs 116 may be used.

In some embodiments, each retention nub comprises a protuberance, a bump, or the like. In some embodiments, each retention nub 116 has an approximate diameter of one (1) millimeter. In other embodiments, each retention nub 116 may be of a different size, be it smaller or larger. The exact size of the retention nubs 116 may vary based on the individual application. For example, an embodiment designed to be used with larger deliver lines may have larger retention nubs, while an embodiment designed to be used with smaller delivery lines may have smaller retention nubs. In addition, in different embodiments, the height of the retention nubs 116 above the surface of the retention channel 104 may vary. In some embodiments, each retention nub is of a uniform size, whereas, in other embodiments, some retention nubs 116 be of a different size than other retention nubs 116 of the plurality of retention nubs 116. The embodiments are not limited to the specific examples provided herein.

Not every embodiment includes retention nubs 116 in every retention channel 104. Some embodiments may include some retention channels 104 with retention nubs 116, while other retention channels 104 may not include retention nubs 116. For example, some applications may require the use of different types of IV lines 106, and an embodiment designed for such an application may include some retention channels 104 with retention nubs 116, and some retention channels without retention nubs 116. Various different combinations are possible, and the embodiments are not limited to the specific examples provided herein.

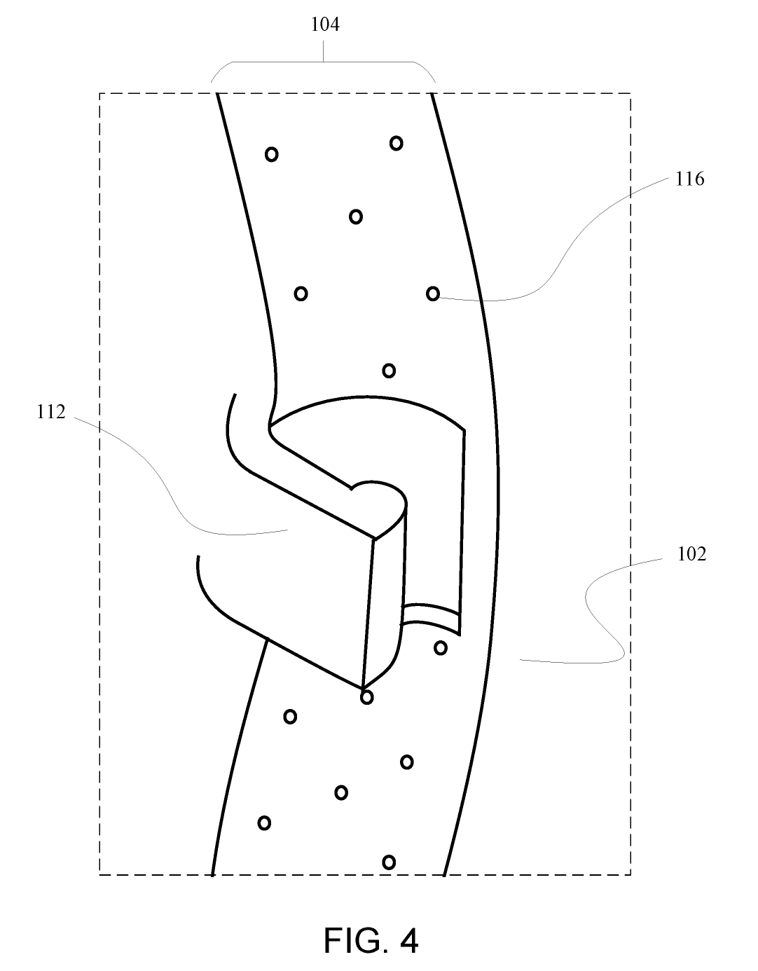

FIG. 4 is a detail view of a portion retention channel 104 of one embodiment of the present disclosure.

In some embodiments, the retention nubs 116 are located along the surface of the retention channel 104 that contacts the IV line 106. In such a location, the retention nubs 116 may sufficiently contact the IV line 106 to aid in retention of the IV line 106. In some embodiments, the plurality of retention nubs 116 may extend onto the surface of the retention tab 112 that contacts the IV line 106. In some embodiments, the retention tab 112 may not bear any of the plurality of retention nubs 116.

The plurality of retention nubs 116 may be arranged in “rows” and “columns” of retention nubs 116. Other embodiments may comprise a “diamond” pattern, or other arrangements of retention nubs 116. Various embodiments may comprise different arrangements of retention nubs 116 along the retention channel, some of which provide for a relatively even spacing between retention nubs 116. Various numbers, arrangements, sizes and other characteristics of retention nubs 116 are also possible, and fall within the scope of the present disclosure. The embodiments are not limited to the specific examples provided herein.

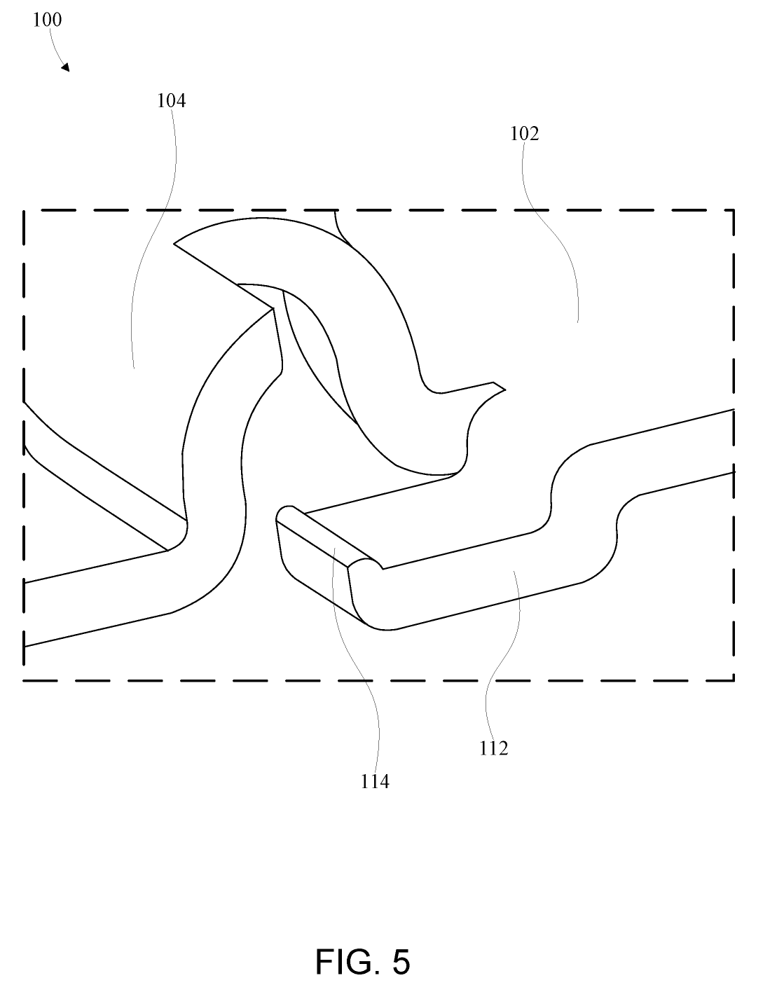

FIG. 5 is a detail view of an end of a retention channel 104 of one embodiment of an intravenous delivery line organizer.

In some embodiments, each retention tab 112 comprises a protrusion 114 that also retains the IV line 106 in the retention channel 104. In some embodiments, the protrusion 114 is present on all retention tabs 112, and in other embodiments, the protrusion 114 may be present only on some retention tabs 112, or not present at all.

The protrusion 114 helps retain the IV line 106 in the retention channel 104 by restricting movement of the IV line 106. When the IV line 106 is placed into the retention channel 104, the user must slightly compress the IV line 106 to push the IV line 106 beyond the protrusion 114. The IV line 106 may then return to its normal shape and be retained by the protrusion 114, the retention tab 112 and the retention channel 104.

In some embodiments, the protrusion 114 is located on the end of the retention tab 112 opposite the end where the retention tab 112 is coupled to the body 102. In some embodiments, the protrusion 114 may run the width of the retention tab 112. In some embodiments, the protrusion 114 may protrude in a direction pointing substantially into the retention channel 104.

In some embodiments, the apparatus 100 comprises a lip running some portion of the length of the retention channel 104 in place of retention tabs 112. The lip performs the same function as the retention tabs 112 (i.e. retention of the IV line 106 within the retention channel 104). In some respects, the lip may be considered to be a modified retention tab 112 that runs the length of the retention channel 104.

In some embodiments, the lip may comprise a protrusion 114 similar to the protrusion 114 described herein, except that the protrusion 114 in such an embodiment runs the entire length of the lip (and therefore the entire length of the retention channel 104).

In some embodiments, the lip may be placed on only one side of the retention channel 104. In other embodiments, a lip may be placed on both sides of the retention channel 104.

Other embodiments may comprise no further retention features beyond the retention channels 104 themselves. In other words, some embodiments may not include retention tabs 112, a lip, or other additional retention mechanism.

In some situations, it may be desirable to organize more IV lines 106 than one apparatus 100 can accommodate. Therefore, in some embodiments, the apparatus 100 comprises connectors at both ends of the body 102. Such an arrangement allows multiple apparatuses to be “daisy-chained” end to end when there is a need to organize more IV lines 106 than one apparatus 100 can accommodate. Different embodiments may use distinct types of connectors, depending upon the situation in which the apparatus 100 is used.

In other embodiments, the connector may be placed at other places on the body 102 of the apparatus 100 to allow for alternate “daisy-chaining” arrangements. The embodiments are not limited to specific configurations or examples provided herein.

In some embodiments comprising connectors, the apparatus 100 is made of a material flexible enough to be curved into a circular shape, and two connectors of the apparatus 100 are coupled to one another to hold the apparatus 100 in a substantially circular shape. Depending on the situation, the apparatus 100 may be curved with the retention channels 104 open towards the interior of the circle formed by the apparatus 100 to help protect the IV lines 106. In other situations, the retention channels 104 may open outwards away from the center of the circle formed by the apparatus 100 for ease of insertion, removal and identification. Both of these circular arrangements reduce the footprint of the apparatus.

In some embodiments, the connectors also allow multiple apparatuses to be coupled to form a polygon shape. Similar to the circular arrangement described herein, in some embodiments the apparatuses may be arranged with the retention channels 104 open towards the interior of the polygon. In other embodiments, the apparatuses may be arranged with the retention channels 104 open away from the center of the polygon. The embodiments are not limited to specific configurations or examples provided herein.

In some embodiments, the apparatus 100 may comprise a mount. In some embodiments, the mount may comprise a clip sized to fit over the railing of a hospital bed or other structure, thereby mounting the apparatus 100 to the hospital bed or other structure. In some embodiments, the clip may provide a retention force to retain the apparatus 100. The retention force may be provided by a spring, the clip itself, or other means. In other embodiments, the clip may simply hang from the hospital bed or other structure.

Other embodiments may comprise a strap or other flexible member to mount the apparatus 100 to any number of objects as the situation may require. For example, some embodiments of the apparatus 100 may be attached to a stand holding an IV pump. In other embodiments, the apparatus 100 may be attached to a stand holding IV bags containing medicines and other liquids that are delivered intravenously. Yet further embodiments may comprise other types of mounts not specifically mentioned herein. The embodiments are not limited to specific configurations or examples provided herein.

In some embodiments, the mount may comprise a plurality of holes in the body 102 through which fasteners may be placed to secure the apparatus 100. In further embodiments, the mount may comprise a single hole through which a fastener may be placed to secure the apparatus 100.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus, comprising:
a body; and
at least one retention channel located in the body that retains an intravenous delivery line;
wherein each retention channel comprises
an open portion that allows placement of the intravenous delivery line into the retention channel, and
at least one retention tab that extends partially across the open portion of the retention channel.

2. The apparatus of claim 1, wherein
at least one retention tab extends from a first side of the retention channel, and
at least one retention tab extends from a second side of the retention channel.

3. The apparatus of claim 1, wherein:
each retention tab comprises a protrusion extending substantially into the retention channel across the open portion of which the retention tab extends.

4. The apparatus of claim 1, wherein:
at least one retention tab extends substantially along a long dimension of its corresponding retention channel.

5. The apparatus of claim 1, wherein at least one retention channel comprises:
a plurality of retention nubs on an interior surface of each retention channel.

6. The apparatus of claim 1, further comprising:
a mount that removably couples the apparatus to an object.

7. The apparatus of claim 6, wherein the object is a rail of a hospital bed.

8. The apparatus of claim 1, wherein each retention channel comprises a first bend.

9. An apparatus, comprising:
a body; and
at least one retention channel located in the body that retains an intravenous delivery line;
wherein each retention channel comprises a first bend.

10. The apparatus of claim 9, wherein
each retention channel comprises an open portion that allows placement of the intravenous delivery line into the retention channel.

11. The apparatus of claim 10, wherein
each retention channel comprises at least one retention tab that extends partially across the open portion of that retention channel.

12. The apparatus of claim 11, wherein
at least one retention tab extends from a first side of the retention channel, and
at least one retention tab extends from a second side of the retention channel.

13. The apparatus of claim 11, wherein:
each retention tab comprises a protrusion extending substantially into the retention channel across the open portion of which the retention tab extends.

14. The apparatus of claim 11, wherein:
at least one retention tab extends substantially along a long dimension of its corresponding retention channel.

15. The apparatus of claim 9, wherein at least one retention channel comprises:
a plurality of retention nubs on an interior surface of each retention channel.

16. The apparatus of claim 9, further comprising:
a mount that removably couples the apparatus to an object.

17. The apparatus of claim 9, wherein:
each retention channel comprises a second bend.

18. A system, comprising:
a plurality of apparatuses, each apparatus comprising
a body, and
at least one retention channel located in the body that retains an intravenous delivery line,
wherein each retention channel of each apparatus comprises
an open portion that allows placement of the intravenous delivery line into the retention channel, and
at least one retention tab that extends partially across the open portion of the retention channel.

19. The system of claim 18, wherein
each of the plurality of apparatuses further comprises at least one connector that removably couples to a connector of another of the plurality of apparatuses; and
at least one apparatus of the plurality of apparatuses is coupled to at least one other apparatus of the plurality of apparatuses.

20. The system of claim 18, wherein at least one retention channel comprises a plurality of retention nubs on an interior surface of the retention channel.
